(19) United States
(12) Patent Application Publication
(10) Pub. No.: US 2009/0287088 A1
(43) Pub. Date: Nov. 19, 2009

(54) LUMINAL CLIP APPLICATOR WITH SENSOR AND METHODS FOR OCCLUDING BODY LUMENS

(75) Inventors: Fred H. Burbank, Laguna Niguel, CA (US); Michael L. Jones, San Clememte, CA (US); R.J. Serra, Irvine, CA (US); Greig E. Altieri, Laguna Beach, CA (US); Jill Ulyeno, Mission Viejo, CA (US); Yu-Tung Wong, Irvine, CA (US); Randy Werneth, Poway, CA (US)

Correspondence Address: Doherty IP Law Group LLC, 37 Belvidere Ave, Washington, NJ, 07882, US

(73) Assignee: Vascular Control Systems, Inc., San Juan Capistrano, CA (US)

(21) Appl. No.: 12/510,909

(22) Filed: Jul. 28, 2009

Related U.S. Application Data
(63) Continuation of application No. 10/300,410, filed on Nov. 19, 2002.
Application No. 10/300,410 is a continuation-in-part of application No. 10/113,096, filed on Mar. 28, 2002, now Pat. No. 7,229,465.
(60) Provisional application No. 60/279,477, filed on Mar. 28, 2001.

(51) Int. Cl. A61B 17/08; A61B 5/0215; A61B 8/14; A61B 17/10

(52) U.S. Cl. 600/465; 606/158; 600/505; 606/142

(57) ABSTRACT

A method for occluding a body lumen includes providing a clamping device having a pair of opposed pressure applying jaws, providing a sensor on one of the pressure applying jaws for sensing blood flow through a body lumen, and releasably securing a luminal clip between the pressure applying jaws. The method includes using the sensor for detecting blood flow through the body lumen for locating the body lumen, positioning the luminal clip adjacent the located body lumen, and closing the pressure applying jaws for clamping the luminal clip over the body lumen for at least partially occluding blood flow through the body lumen.

### CROSS-REFERENCE TO RELATED APPLICATIONS

This application is a continuation of U.S. patent application Ser. No. 10/300,410, filed Nov. 19, 2002, which is a continuation-in-part of U.S. patent application Ser. No. 10/113,096, filed Mar. 28, 2002, which claims the benefit of U.S. Provisional Application Ser. No. 60/279,477, filed Mar. 28, 2001, which applications are hereby incorporated by reference herein in their entirety and from which priority is hereby claimed under 35 U.S.C. § 119(e) and 35 U.S.C. § 120.

### FIELD OF THE INVENTION

The present invention relates generally to the field of medical devices and treatments of diseases and conditions by the detection and occlusion of internal body lumens such as blood vessels and ducts by application of luminal clips.

### BACKGROUND OF THE INVENTION

Many surgical procedures, including tubal ligation, cholecystectomy, appendectomy, liver biopsy, and other procedures, often require the placement of a luminal clip, typically to occlude a blood vessel, duct, or other vessel, or to close an incision. For example, removal of a gall bladder requires that the cystic duct and the cystic artery feeding the gall bladder be cut and then closed, typically with a luminal clip, to prevent leakage of blood or bile, and to promote healing of the cut artery and duct. However, it is often difficult to distinguish between blood vessels, ducts, and other tissues, and to identify the proper tissue to which to apply a luminal clip.

Hysterectomy (surgical removal of the uterus) is performed on approximately 600,000 women annually in the United States. Hysterectomy is often the therapeutic choice for the treatment of uterine cancer, adenomyosis, menorrhagia, prolapse, dysfunctional uterine bleeding (abnormal menstrual bleeding that has no discrete anatomic explanation such as a tumor or growth), and muscular tumors of the uterus, known as leiomyoma or uterine fibroids.

However, hysterectomy is a drastic treatment, having many undesirable characteristics. Thus, any method which can approximate the therapeutic result of a hysterectomy without removing the uterus would be a significant improvement in this field. Newer treatment methods have been developed for some diseases which may spare these women a hysterectomy.

In 1995, it was demonstrated that uterine fibroids could be treated without hysterectomy using a non-surgical therapy, specifically comprising bilateral intraluminal occlusion of the uterine arteries (Ravina et al., “Arterial Embolization to Treat Uterine Myomata”, Lancet Sep. 9, 1995; Vol. 346; pp. 671-672, incorporated in its entirety herein). This technique is known as “uterine artery embolization”. In this technique, the uterine arteries are accessed via a transvascular route from a common femoral artery into the left and right uterine arteries.

The uterus has a dual (or redundant) blood supply, the primary blood supply being from the bilateral uterine arteries, and the secondary blood supply from the bilateral ovarian arteries. Consequently, when both uterine arteries are occluded, i.e. bilateral vessel occlusion, the uterus and the fibroids contained within the uterus are both deprived of their blood supply. However, as demonstrated by Ravina et al., the effect on the fibroid is greater than the effect on the uterus. In most instances, the fibroid withers and ceases to cause clinical symptoms.

However, many physicians do not possess the skill or equipment necessary to perform catheter-based uterine artery embolization under radiologic direction. Accordingly, only thousands of uterine artery embolizations have been performed, worldwide, over the past three years, whereas hundreds of thousands of hysterectomies have been performed each year for uterine fibroids which are symptomatic.

What is needed, therefore, are devices and methods to detect vessels and to place clips onto vessels so as to occlude vessels such as the cystic duct and the uterine arteries that can be used by physicians of ordinary skill in a simple medical setting or environment.

### SUMMARY OF THE INVENTION

The invention is directed to detecting and occluding internal body lumens, such as blood vessels, ducts, lymph nodes and other lumens, and specifically, the uterine arteries of a female patient. In particular, the invention provides clip applicators configured to apply luminal clips to body lumens. Application of a luminal clip onto or around an internal body lumen is typically sufficient to compress and at least partially collapse the body lumen so as to reduce or abolish fluid flow through the vessel. Clip applicators having features of the invention have a sensor or sensors effective to detect an internal body lumen such as a blood vessel or a duct. Clip applicator devices may gain access to internal body lumens via surgical incisions, or may indirectly access internal body lumens via body cavities and orifices.

A system having features of the invention includes a luminal clip applicator having a sensor and a signal conductor. Systems may further include a luminal clip, and/or a sensor controller. The sensor is configured to detect a body lumen such as a blood vessel, preferably by sensing blood flow, and is configured to operatively connect to a sensor controller via a signal conductor that is configured to carry signals from the sensor to the sensor controller. A signal conductor may also carry power, control signals, and other signals to the sensor. In embodiments of the invention, the signal conductor is an integral part of the luminal clip applicator, and may be permanently or transiently connected to a sensor controller. A sensor controller is configured to receive signals from a sensor, and may provide sensor signal outputs for interpretation by an operator. A sensor controller may also supply power and signal energy to a sensor.

A luminal clip applicator embodying features of the invention has at least one, and preferably a pair of pressure-applying members disposed on at least one extension member supporting the pressure-applying members, and a sensor. A sensor may sense blood flow, sound, pulsation, pH, or other indicator related to a body lumen, which may be used to locate a body lumen such as a blood vessel. The sensor may be disposed on a pressure-applying member. A pressure-applying member has a pressure-applying surface configured to hold and to compress a luminal clip onto or around a body lumen effective to occlude it. For example, the pressure-applying members may include a pair of jaws joined at a pivot configured to allow the jaws to move so as to clamp onto a luminal clip held within the jaws, thereby compressing the clip. A compressed luminal clip, when placed around a vessel, or onto tissue surrounding a vessel, may be effective to occlude the vessel. Occlusion may be temporary or permanent, and may be partial or complete, so that flow of fluid (such as blood, bile, lymph or other fluid) through the lumen (e.g., a blood vessel or duct) is reduced, or is abolished, by compression of a luminal clip around the lumen. A nitinol, stainless steel or titanium clip, for example, may occlude a blood vessel or duct indefinitely, while biodegradable clips may be used to temporarily occlude a blood vessel or duct.

A luminal clip suitable for use with a luminal clip applicator having features of the invention is configured to engage a body lumen, preferably by at least partially encircling it, and to compress it. Such compression is effective to at least partially occlude it. A luminal clip typically has an open configuration before application, and a closed configuration after application. Such a clip may include loops or coils of metal, polymer, or other deformable material; flexible or moveable bars or bands, which may be joined by a hinge or hinges; and snaps or other capture elements configured to retain a clip in a closed configuration. A clip may be made with bio-resorbable materials, metal, including “memory” materials such as nickel titanium alloys or other memory metals, or other biocompatible materials.

Luminal clip applicators with sensors embodying features of the invention may be used to apply luminal clips to occlude body lumens, providing a single device or system configured for both the detection of target body lumens and for the placement of luminal clips onto them. Unlike prior art clip applicators, such a luminal clip applicator with a sensor facilitates the accurate deployment of the clip and provides the advantage of being able to differentiate between a duct and a blood vessel. This minimizes the risk of misapplication of a luminal clip. These devices and methods for applying luminal clips are simpler and more readily used than other methods and devices, and provide improved treatments for serious conditions and diseases, including uterine fibroids, adenomyosis, dysfunctional uterine bleeding (DUB), postpartum hemorrhage, and for other uterine disorders. Devices, systems and methods embodying features of the invention provide tools and methods for effective treatment of diseases and conditions that might otherwise require invasive and irreversible treatments such as removal of a uterus.

### BRIEF DESCRIPTION OF THE DRAWING

FIG. 1 is a plan view of a system embodying features of the invention including a luminal clip applicator embodying features of the invention disposed in an open configuration.

FIG. 2 is a plan view of a distal portion of a luminal clip applicator embodying features of the invention disposed in an open configuration.

FIG. 3 is a plan view of a distal portion of a luminal clip applicator embodying features of the invention disposed in an open configuration and containing an open luminal clip.

FIG. 4 is a plan view of a distal portion of a luminal clip applicator embodying features of the invention disposed in a closed configuration and containing a closed luminal clip.

FIG. 5 is a transverse cross-sectional view of a jaw portion of the luminal clip applicator and clip of FIG. 3 taken at line 5-5.

FIG. 6 is a transverse cross-sectional view of a jaw portion of the luminal clip applicator and clip of FIG. 4 taken at line 6-6.

FIG. 7 is a perspective view of a clip suitable for use with a luminal clip applicator having features of the invention.

FIG. 8 is a schematic diagram illustrating the use of a luminal clip applicator embodying features of the invention in the occlusion a uterine artery of a female human patient.

FIG. 9 is a schematic diagram illustrating the use of a luminal clip applicator embodying features of the invention in the occlusion a bile duct of a human patient.

### DETAILED DESCRIPTION

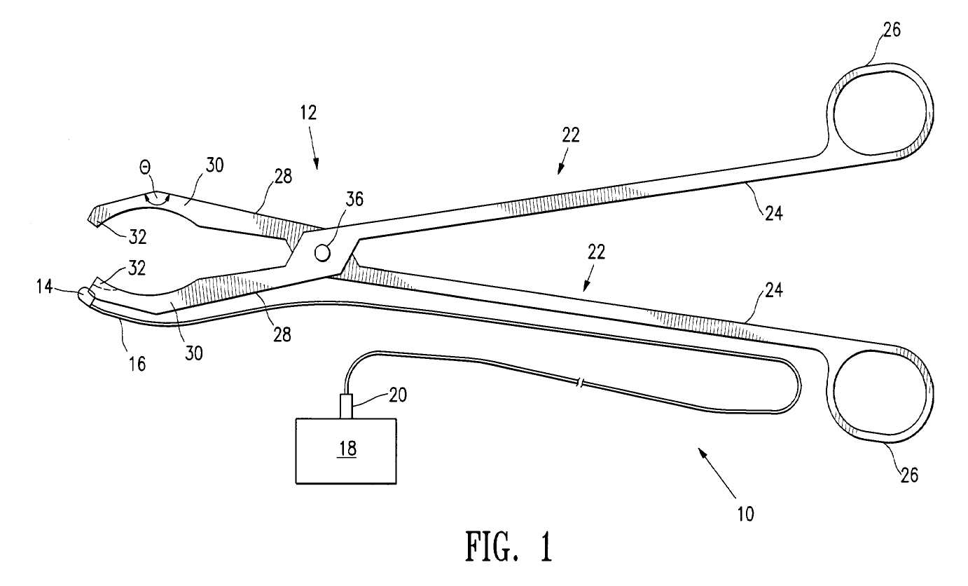

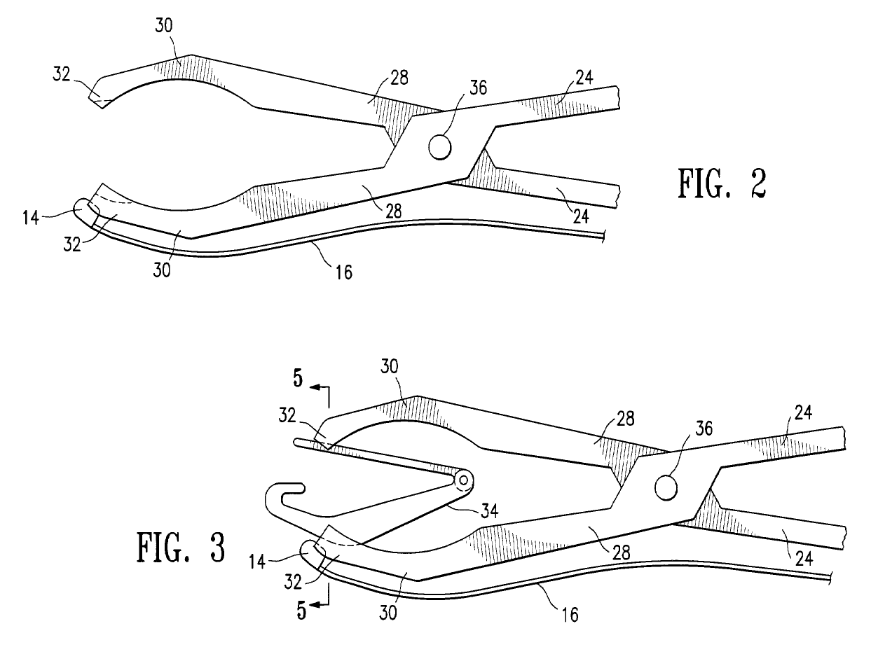

FIGS. 1-6 show a luminal clip applicator system 10 embodying features of the invention. The system 10 includes a clip-applying device 12, a body lumen sensor 14 and an energy transmission member 16, e.g. a conductor, connected to the body lumen sensor 14 and configured to be operatively connected to a sensor controller 18 (e.g., via a connector 20).

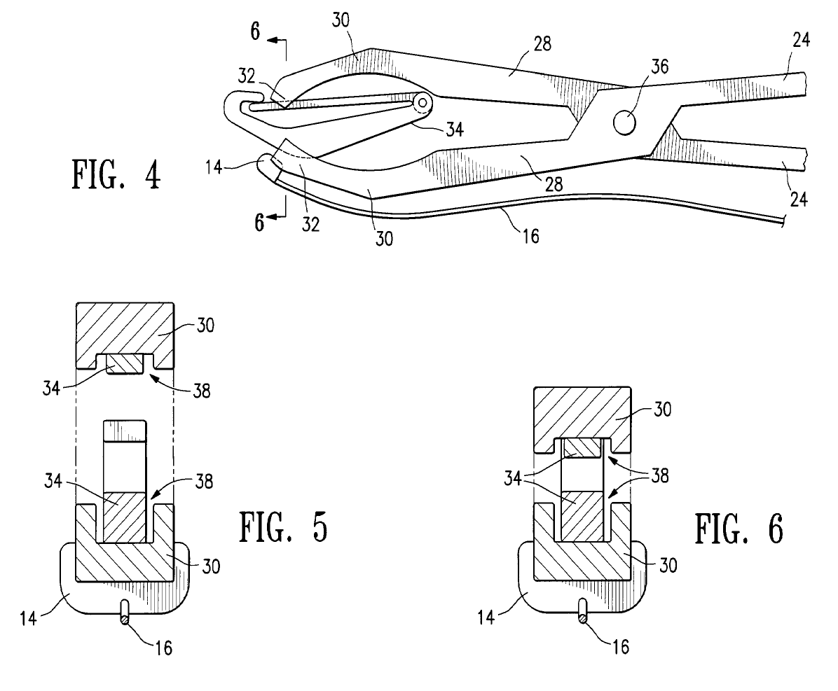

The clip-applying device 12 has elongated members 22 having proximal handle portions 24 with finger holes 26 and distal jaw portions 28 with jaws 30. Jaws 30 have pressure-applying surfaces 32 configured to engage and hold a luminal clip 34 (e.g., as shown in FIGS. 3-6). Elongated members 22 are pivotally connected at pivot point 36 located proximal to the jaws 30, so that squeezing proximal handle portions 24 together closes jaws 30, compressing a luminal clip 34 as shown in FIGS. 4-6. A jaw 30 and a pressure-applying surface 32 may have a clip-engaging feature 38, such as a slot or ridge, or other feature configured to releasably retain a luminal clip 34 in place before it is clamped onto a body lumen, while allowing its ready release from the jaws 30 of the clip-applying device 12 after it has been compressed onto a body lumen. In further embodiments, applicators 12 having features of the invention may also have a mechanism, such as a ratchet mechanism, configured to releasably hold elongated members 22 in a closed or partially closed configuration.

The sensor 14 is operatively connected by energy transmission member 16 to sensor controller 18. Sensor 14 may be any suitable sensor for directly or indirectly detecting a body lumen, and is preferably a Doppler ultrasound sensor. The energy transmission member 16 may include an electrical conductor, an optical fiber, an optical waveguide, or other conduit for carrying energy or signals to or from a sensor 14. A connector 20 is preferably configured to readily engage and disengage with a sensor controller 18, although connector 20 may form a permanent engagement with sensor controller 18. Alternatively, energy transmission member 16 may be directly and permanently secured by soldering, brazing or the like to sensor controller 18 without having a connector 20. A sensor controller 18 may supply power to a sensor 14, provide signal energy to a sensor 14, regulate the operation of a sensor 14, receive signals from a sensor 14, and may provide sensor signal outputs for interpretation by an operator.

Thus, luminal clip applicators embodying features of the invention are configured to place a luminal clip 34 around a body lumen and to apply pressure effective to clamp the luminal clip 34 onto the lumen or onto tissue near to it, occluding it, preferably without damaging the lumen wall. Preferably, the amount of force applied to a luminal clip 34 placed by an applicator device 12 embodying features of the invention is between about 1 pound and about 20 pounds, preferably between about 2 pounds and about 10 pounds, more preferably between about 6 and about 9 pounds. Less force is needed to completely occlude blood flow when a clip is applied directly to a blood vessel (e.g., as low as about 3 psi), while greater force may be necessary to completely occlude blood flow in a blood vessel when a clip is applied to tissue surrounding a blood vessel and not directly to it. Such force is effective to apply pressure to a luminal clip so as to compress it onto or around tissue to occlude a body lumen held within a luminal clip 34. The amount of pressure delivered by such force depends upon the surface area in contact with the luminal clip 34; however, the amount of pressure applied to a luminal clip 34 placed by an applicator device 12 embodying features of the invention is typically between about 3 pounds per square inch (psi) and about 200 psi. A suitable luminal clip 34, when clipped around a body lumen, is effective to apply pressures of between about 5 pounds per square inch (psi) and about 80 psi, preferably about 7 psi and about 10 psi to a lumen.

A distal jaw portion 28 may have a longitudinal dimension, such as a length, sized to deliver a jaw 30 of a clip applicator device 12 to a desired internal region of a patient's body while providing sufficient distance between the point of application of a clip 34 and the proximal ends of elongated members 22 as to allow ready use of the applicator 12 in a medical procedure (e.g., by providing sufficient distance (e.g., between about 3 inch and about 8 inch) between jaws 30 and finger holes 26 as to allow an operator to manipulate the applicator 12 while applying jaws 30 to a body lumen through an incision through the skin of a patient). Such a longitudinal dimension may comprise a distance of between about 2 cm and about 20 cm, preferably between about 3 cm and about 15 cm. A jaw 30 of an applicator device 12 embodying features of the invention may have a length of between about 0.3 cm and about 10 cm, preferably between about 1 cm and about 8 cm.

A jaw 30 may be configured to join with a distal jaw portion 28 on a line substantially parallel to a line along the distal jaw portion 28, or may join at an angle to such a line. An angle θ between a jaw 30 and a distal jaw portion 28 may be acute or may be obtuse. For example, where an angle θ is not 180°, an angle θ between a jaw 30 and a distal jaw portion 28 may be about a 90° angle, may be about a 120° angle, or may be about a 135° angle.

The jaws 30 and/or distal jaw portion 28 of an applicator device 12 may be hinged, as by a pivot 36, effective to allow opposed jaws to close together, thereby compressing a luminal clip 34 around a blood vessel or other body lumen located between jaws 30. Alternatively, a single movable jaw 30 may close onto an anvil, thereby compressing a luminal clip 34 between the jaw 30 and the anvil. Thus, a pressure-applying member may be a jaw 30, an anvil, or other member, and a pressure-applying surface 32 may be a surface of a jaw 30, a surface of an anvil, or other surface. Any suitable mechanism for compressing a luminal clip 34 may be used.

A sensor 14 may include any sensor for locating a body lumen, including a sensor configured for detecting blood flow. A sensor 14 may be attached to a distal jaw portion 28, and is preferably mounted on a jaw 30. For example, a blood flow sensor may be mounted between about 0.1 inch and about 1 inch from the distal tip of a jaw 30, and is preferably mounted about 0.2 inch to about 0.6 inch, more preferably about 0.4 inch from the distal tip of a jaw 30. Alternatively, a sensor 14 may be mounted on a support disposed near a jaw 30 or distal jaw portion 28, or otherwise mounted on a clip-applying device 12 effective to sense a body lumen.

A sensor 14 may be passive (detecting intrinsic signals indicating the presence of a blood vessel or other lumen) or active (producing a signal and detecting a response to it). A sensor 14 may thus be a microphone (e.g., to sense heart sounds), an ultrasound sensor, a pressure transducer, a stress gauge or strain gauge to detect pulsations in a blood vessel due to heart action, an electromagnetic sensor (e.g., infrared sensor) to detect a blood vessel (e.g., to detect hemoglobin), a pH or other chemical sensor, or other sensor. Preferably, a sensor 14 is a Doppler ultrasound sensor, configured to emit and to detect ultrasound signals effective to detect blood flow and to locate a blood vessel.

Doppler ultrasound uses ultrasound signals reflected by moving blood cells in a blood vessel to measure blood flow. Ultrasound reflected back towards the ultrasound crystal typically has a frequency shift, that is, returns to the crystal at a different frequency than the frequency emitted by the ultrasound crystal. Ultrasound reflected back from blood cells moving away from the crystal has a lower frequency than the source ultrasound frequency; ultrasound reflected back from blood cells moving towards the crystal has a higher frequency than the source frequency. This Doppler frequency shift phenomenon can be measured by the transceiver electronics and sent to the speaker to create sounds detectable by an operator. The speaker sound output may be configured to correspond to the velocity of the moving blood cells: for example, the sound output may change in pitch (frequency) as the blood cell velocity changes; the volume of the sound output may change (i.e., a loud speaker signal may be used to indicate a big Doppler shift corresponding to a fast velocity, while a fainter speaker output signal may be used to indicate a small Doppler shift corresponding to slow velocity of blood flow); or another such sound signal may be used to indicate a change in the velocity of the moving blood cells.

In general, preferred Doppler devices utilize either pulsed or continuous wave Doppler signals generated at a frequency suitable for detecting blood flow in nearby vessels, and have a relatively narrow field of view and limited depth of view so as not to generate a signal from other blood vessels. Commercially available Doppler ultrasound sensor systems suitable for use in the present invention include the Koven model ES 100X MiniDop VRP-8 probe (St. Louis, Mo.), the DWL/Neuro Scan Medical Systems' Multi-Dop B+ system (Sterling, Va.), and the MedaSonics® CardioBeat® Blood Flow Doppler with Integrated Speaker (Cooper Surgical, Inc., Trumbull Conn. 06611)). Along with the Doppler sensor, these sensor systems include a sensor controller and a conductor connecting the sensor and the sensor controller.

A sensor controller 18 such as a Doppler ultrasound sensor controller may include an electrical connector to plug in the location sensor, a power switch to power-on the transceiver electronics, an audible speaker output so that an operator can hear the Doppler frequency shift, a volume adjustment to control overall sound level, and batteries or other power source to provide energy. For example, a sensor controller 18 having an energy source may provide electrical energy which aids a piezoelectric ultrasound sensor to produce and to detect ultrasound energy.

To detect blood flow in the uterine arteries, ultrasound transducers may be placed within a vagina. Ultrasound transducers may, for example, point axially into the patient's tissue and insonate it to a depth of typically 2 cm (attenuated through tissue) for 8 MHz systems. The bilateral uterine arteries run laterally inward from sidewall of pelvis to the uterus just behind the vaginal mucosa near the cervix, and are by far the single largest blood vessels in this area, making their detection by ultrasound relatively straightforward. In addition, the inventors have discovered that a Doppler crystal may be optimized for uterine vessel detection by configuring it to detect blood flow in a wide region detected by the location sensors.

Ultrasound frequencies suitable for use with a Doppler ultrasound sensor include frequencies between about 5 Megahertz (MHz) and about 20 MHz, preferably between about 6 MHz and about 10 MHz, more preferably about 8 MHz. A sensor 14 may also be, for example, an infrared sensor or other electromagnetic sensor. Electromagnetic energy useful for sensing a location of a blood vessel or of blood flow in a blood vessel may be infrared energy having a wavelength of between about 500 nanometers (nm) and about 2000 nm, preferably between about 700 nm and about 1000 nm.

Doppler ultrasound devices typically have an ultrasound dispersion pattern which is slightly conical, so that a sensor 14 typically “looks” out along a cone-shaped “field of view” (the solid angle within which ultrasound signals may be detected) generally around a line centered within the solid angle which is the preferred direction from which signals may be received by a sensor 14. The solid angle may be wide or narrow. The preferred direction may be perpendicular, parallel, or at another angle to a surface in or on which the sensor 14 is located. Thus, a sensor 14 preferably has a sensing direction, in which a blood vessel that is located along a sensing direction is detectable by the sensor 14. A sensing direction is defined with respect to a sensor 14, and typically includes a range of directions, such as a range of directions within a solid angle taken with respect to a pressure-applying surface 32 of a jaw 30 in or on which a sensor 14 is disposed, effective that a body lumen (such as a blood vessel) disposed at least in part in or across the solid angle of a sensing direction is detectable by a sensor 14. Thus, a sensor 14 may be configured to indicate the location of a blood vessel with respect to a jaw 30. For example, a sensor 14 may be oriented perpendicularly to the pressure-applying surface 32, so that sensor 14 is typically disposed on or within a jaw 30 with its sensing direction is substantially perpendicular to a pressure-applying surface 32 of a jaw 30. Such a sensing direction is effective to locate blood vessels or detect blood flow in arteries facing a jaw 30, or between jaws 30. A sensing direction that is substantially perpendicular to a pressure-applying surface 32 may include directions from a sensor 14 within a solid angle of between about 70° and about 110° with respect to the pressure-applying surface 32. However, other sensing directions are also suitable, including, for example, sensing directions that are substantially parallel to a pressure-applying surface 32.

A sensor 14 is preferably a Doppler ultrasound sensor 14. A Doppler ultrasound sensor 14 can be configured to identify blood vessels located within a predetermined distance range, e.g. from between about 0 cm and about 5 cm, more preferably between about 0.5 cm and about 3 cm, from the sensor 14. Thus, lumens with fluid flow in them, such as a blood vessel, which lie generally in a preferred direction from a sensor 14, and within the effective range of the sensor 14, would be detected by the sensor 14. Presently available Doppler ultrasound devices may use pulsed or continuous ultrasound, and are typically attached to a signal processing and display unit, which may serve as a sensor controller 18, which processes the electrical signals generated by a Doppler device to generate displays and/or other data derived from the electrical signals for additional uses.

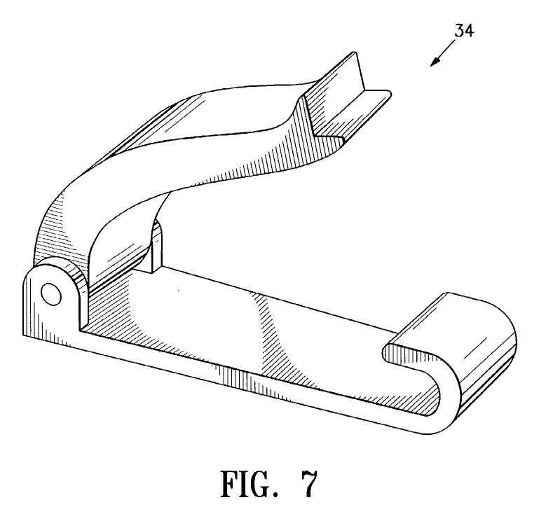

An example of a luminal clip suitable for use with a luminal clip applicator 12 having features of the invention is shown in an open configuration in FIG. 3, and in a closed configuration in FIG. 4. Typically, a clip 34 has two arms which can be separated in an open configuration, and can be disposed near to each other in a closed configuration. Alternatively, a clip may be formed from a single piece of material that can be maneuvered or deformed so as to compress a body lumen, or a clip may be made from multiple pieces of material. A further example of a luminal clip suitable for use with a luminal clip applicator 12 having features of the invention is shown in FIG. 7. As shown in FIG. 7, a clip 34 may have a curved arm and a flat arm joined by a hinge at one end. In an open configuration, the arms are separated at an end opposite the hinge. The arms may be secured in the closed configuration by, for example, securing a tab beneath a lip (as shown, e.g., in the example illustrated in FIG. 4).

Methods and devices embodying features of the invention may be used to place a luminal clip onto or around any body lumen effective to occlude it. For example, a uterine artery or a cystic duct may be occluded by clamping a luminal clip onto the uterine artery or cystic duct, or onto adjacent tissue, effective to occlude the uterine artery or cystic duct. Occlusion of a uterine artery may be used to treat uterine disorders such as, for example, uterine fibroids, dysfunctional uterine bleeding, and other uterine disorders. Removal of a gall bladder typically requires the occlusion of the cystic duct, preferably with luminal clips. It will be understood that the methods and devices discussed in regard to this example may also be applied to any other artery, vein, duct, node, or other lumen.

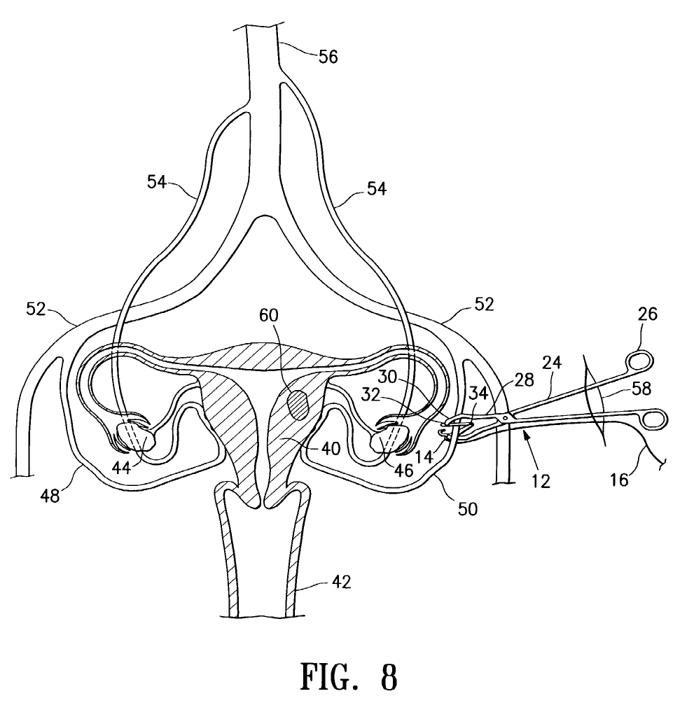

FIG. 8 illustrates the use of a luminal clip applicator embodying features of the invention. A typical human female reproductive system is shown, including a uterus 40, vagina 42, right ovary 44, and left ovary 46. Blood is supplied to the uterus 40 primarily via the right uterine artery 48 and the left uterine artery 50, branching off the iliac arteries 52, and secondarily via the ovarian arteries 54, which branch off from the aorta 56.

A luminal clip applicator 12 with a sensor 14 carrying a luminal clip 34 may be introduced into a patient's body via an incision 58 and used to detect and/or locate a body lumen such as a blood vessel or duct, and/or to occlude the blood vessel or duct by clamping a luminal clip 34 onto it. For example, a luminal clip applicator 12 embodying features of the invention may used to sense the location of a uterine artery 48 or 50, and may be used to apply a luminal clip 34 effective to compress and to occlude a uterine artery effective to reduce or abolish blood flow in that uterine artery.

The uterus 40 illustrated in FIG. 8 has a uterine fibroid 60 (one of several medical conditions which may be treated by occlusion of the uterine arteries). The uterine arteries 48 and 50 approach the uterus 40 not far from the vagina 42.

A luminal clip applicator 12 is shown disposed partly within the abdominal cavity of the patient, having gained access to the uterine arteries 48 or 50 via the incision 58. Alternatively, where a vessel is accessible via a body cavity or body orifice, a luminal clip applicator 12 may access a target body location without going through an incision 58. Jaws 30 holding luminal clip 34 are shown disposed around a left uterine artery 50. Jaws 30 have pressure-applying surfaces 32 holding luminal clip 34 in place in contact with left uterine artery 50. Alternatively, luminal clip 34 may be in contact with tissue around left uterine artery 50.

The luminal clip applicator 12 also includes a sensor 14 on a jaw 30 facing the patient's tissue, and communicating with other parts of the system 10 via energy transmission member 16. The sensor 14 is useful in locating a uterine artery 48 or 50, and may detect blood flow in the arteries to help differentiate a blood vessel from a duct or other anatomical structure. Once a luminal clip applicator 12 has been positioned near a uterine artery located with sensor 14, a luminal clip 34 may be placed around the artery and clipped to the artery, occluding it. Blood flow in the artery may be detected with a sensor 14, and the reduction or cessation of blood flow though the occluded artery may be detected by sensor 14.

Blood flow in the right uterine artery 48 may be similarly occluded, by placement of another clip 34 by the same luminal clip applicator 12 (after placement of a luminal clip 34 onto the left uterine artery 50) or by a different luminal clip applicator 12 (thus allowing simultaneous clamping and occlusion of both uterine arteries).

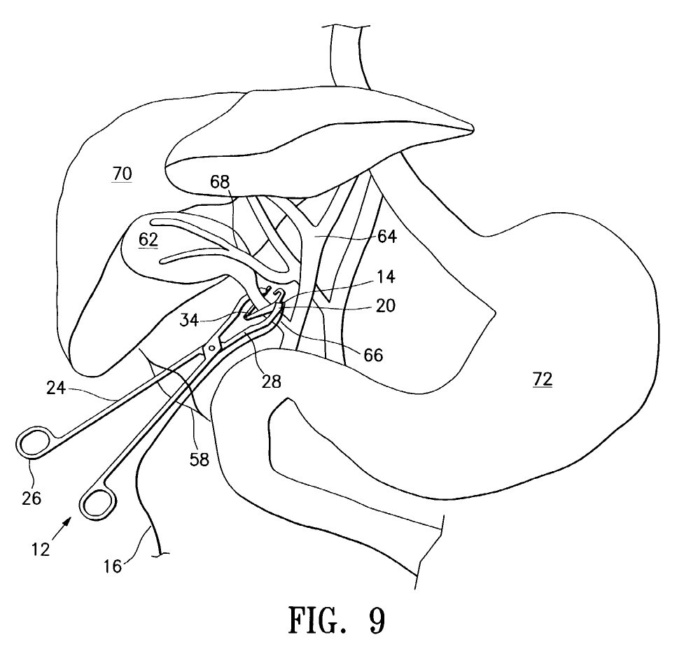

Luminal clip applicator devices and methods embodying features of the invention may be used to occlude body lumens that are not blood vessels, such as ducts and lymph nodes, as well as to occlude blood vessels. For example, luminal clip applicator devices and methods embodying features of the invention may be used to occlude a cystic duct. Shown in FIG. 9 is a schematic diagram of an abdominal portion of a human patient showing the gall bladder 62 and bile duct 64, which are connected by the cystic duct 66. The cystic artery 68 is located near to the cystic duct 66. The liver 70 and stomach 72 are also shown in FIG. 9.

FIG. 9 illustrates the use of a luminal clip applicator 12 embodying features of the invention in the occlusion a cystic duct 66 of a human patient, as may be performed during a cholestectomy procedure. A luminal clip applicator 12 may be inserted into a patient's abdominal cavity via an incision 58 and guided towards cystic duct 66. The luminal clip applicator 12 has distal jaw portions 28 disposed at least partially within the patient's body, positioning jaws 30 holding luminal clip 34 near a cystic duct 66. Jaws 30 are shown disposed around a cystic duct 66. Sensor 14 may be used in the location of a cystic duct 66, for example, by being used to differentiate cystic duct 66 from nearby cystic artery 68. For example, detection of blood flow with a blood flow sensor 14 indicates that sensor 14 (and therefore jaws 30) is adjacent a cystic artery 68 and not the target cystic duct 66. Once the cystic duct 66 as been identified, visual observation and sensor 14 may be used to guide the placement of jaws 30 and luminal clip 34 (carried in jaws 30) around the cystic duct 66. Moving distal jaw portions 28 so as to compress jaws 30 around clip 34 is effective to clamp luminal clip 34 around the cystic duct 66, occluding it.

Thus, a method of occluding a body lumen includes sensing a body lumen, and compressing a body lumen with a luminal clip applied by a device having a sensor. A method of occluding a body lumen includes detecting and/or locating a body lumen with a sensor, and compressing a body lumen with a luminal clip placed by a device having a sensor. Detecting and/or locating a body lumen may include positive detection and/or location, as by detecting and/or locating a blood vessel by detecting blood flow in the blood vessel. For example, a method of occluding a uterine artery includes detecting and/or locating a uterine artery with a sensor, and compressing a uterine artery with a luminal clip placed by a device having a sensor. Detecting and/or locating a body lumen may include negative detection and/or location in which a duct or lymph node is differentiated from a blood vessel by a sensor. For example, a cystic duct may be detected and/or located by identifying the nearby cystic artery by detecting blood flow in the cystic artery, and avoiding the cystic artery.

A luminal clip suitable for use in a method embodying features of the invention may be a releasable clip, so that a uterine artery may remain occluded for only a limited time. A releasable clip may be mechanically releasable, may be bioabsorbable, or otherwise configured to release pressure on a lumen after a period of occlusion. For example, a bioabsorbable clip, which is degraded and/or absorbed by a patient's body over time after placement on a vessel, provides a temporary occlusion that remains for only a limited time. A suitable limited time for clips applied to a cystic duct may be, for example, about 6 months, or preferably about 6 weeks, or more preferably about 2 weeks, or yet more preferably about 1 week. A suitable limited time for clips applied to a uterine artery to treat uterine fibroids may be, for example, between about 0.5 hours and about 1 week, or preferably between about 0.5 hours and about 12 hours, or more preferably between about 1 hour and about 8 hours.

A device or system embodying features of the invention may be designed for single use (disposable) or may be sterilizable and capable of multiple use. Suitable materials for use in making a luminal clip applicator embodying features of the invention include metals such as stainless steel, ceramics, plastics, and other materials. Biocompatible polymers, such as for example, polycarbonate, polysulfone, polyester, polyethylene, polyacetal, and other polymers may be particularly suitable for use in making a luminal clip applicator devices embodying features of the invention.

Luminal clips for use with clip applicators embodying features of the invention may be made from any suitable material or combination of materials, including metals such as stainless steel, tantalum, titanium, and shape memory alloys such as nickel titanium alloys, ceramics, polymers such as polyesters, polyamides, polycarbonates, polyvinyl chloride, polysulfones, polyacetals and polypropylenes, and other materials known in the art. Luminal clips made of such materials typically remain in place permanently after application. Bioabsorbable luminal clips may be made from, for example, bioabsorbable polymers such as homopolymers and copolymers of lactide, glycolide, caprolactone and dioxanone compounds such as poly(p-dioxanone).

While particular forms of the invention have been illustrated and described, it will be apparent that various modifications can be made without departing from the spirit and scope of the invention. Accordingly, it is not intended that the invention be limited to the specific embodiments illustrated. It is therefore intended that this invention be defined by the scope of the appended claims as broadly as the prior art will permit, and in view of the specification if need be. Moreover, those skilled in the art will recognize that features shown in one embodiment may be utilized in other embodiments. Terms such a “element”, “member”, “device”, “sections”, “portion”, “section”, “steps” and words of similar import when used herein shall not be construed as invoking the provisions of 35 U.S.C. § 112(6) unless the following claims expressly use the terms “means” or “step” followed by a particular function without specific structure or action. All patents and patent applications referred to above are hereby incorporated by reference in their entirety.

What is claimed is:

1. A method of occluding a body lumen of a patient comprising:
locating a body lumen with a sensor disposed on a vessel-occlusion device carrying a body lumen occluding clip; and
compressing a portion of said body lumen with said body lumen occluding clip applied to said vessel by said vessel-occlusion device comprising said sensor.

2. The method of claim 1, wherein said body lumen is selected from the group of body lumens consisting of blood vessels, ducts, and lymph nodes.

3. The method of claim 1, wherein said body lumen is a blood vessel.

4. The method of claim 1, wherein said sensor comprises a blood flow sensor and the locating said body lumen step comprises detecting blood flow in a blood vessel.

5. The method of claim 4, further comprising detecting a reduction in blood flow in said blood vessel.

6. The method of claim 4, wherein said blood flow sensor comprises a Doppler ultrasound blood flow sensor.

7. The method of claim 6, wherein locating with said Doppler ultrasound blood flow sensor comprises locating said body lumen with ultrasound having a frequency of between about 5 MHz and about 20 MHz.

8. The method of claim 3, wherein said blood vessel is a uterine artery.

9. The method of claim 8, wherein said sensor comprises a blood flow sensor and said locating comprises detecting blood flow in a uterine artery.

10. The method of claim 9, further comprising detecting a reduction in blood flow in said uterine artery.

11. The method of claim 1, wherein said body lumen occluding clip comprises a releasable clip configured to release said occlusion after a limited time.

12. The method of claim 11, wherein said limited time comprises a time of between about 0.5 hour and about 6 months.

13. The method of claim 1, wherein said body lumen occluding clip comprises a releasable clip configured to compress tissue for a limited time comprising between about 0.5 hour and about 1 week.

14. The method of claim 1, wherein said body lumen occluding clip comprises a releasable clip configured to compress tissue for a limited time comprising between about 0.5 hour and about 12 hours.

15. The method of claim 14, wherein said limited time comprises a time period of between about 1 hour and about 8 hours.

16. A method of occluding a body lumen comprising:
providing a clamping device having a distal end including first and second pressure applying jaws with opposing luminal clip receiving surfaces;
providing a sensor on at least one of said pressure applying jaws for sensing the location of one or more body lumens;
releasably securing a luminal clip between said opposing luminal clip receiving surfaces;
using said sensor for sensing the location of a body lumen;
closing said pressure applying jaws for compressing said body lumen with said luminal clip so as to at least partially occlude said body lumen.

17. The method as claimed in claim 16, wherein said body lumen is a uterine artery and said sensor is a blood flow sensor.

18. The method as claimed in claim 16, wherein said luminal clip is bioabsorbable for releasing compression on said body lumen after a time period of between about 0.5 hours and 6 months.

19. A method for occluding a body lumen comprising:
providing a clamping device having a pair of opposed pressure applying jaws;
providing a sensor on one of said pressure applying jaws for sensing blood flow through a body lumen;
releasably securing a luminal clip between said pressure applying jaws;
using said sensor for detecting blood flow through said body lumen for locating said body lumen;
positioning said luminal clip adjacent said located body lumen;
closing said pressure applying jaws for clamping said luminal clip over said body lumen for at least partially occluding blood flow through said body lumen.

20. The method as claimed in claim 19, further comprising after a time period, unclamping said luminal clip for restoring normal blood flow through said body lumen.

21. The method as claimed in claim 20, wherein said luminal clip is bioabsorbable, and wherein the unclamping said luminal clip comprises absorbing said luminal clip within a patient's body.
